(19) United States
(12) Patent Application Publication
(10) Pub. No.: US 2007/0016177 A1
(43) Pub. Date: Jan. 18, 2007

(54) Laser ablation apparatus useful for hard tissue removal

(76) Inventors: Boris Vaynberg, Zichron Ya'akov (IL); Yotam Zimmerman, Hadera (IL)

Correspondence Address: DEKEL PATENT LTD., DAVID KLEIN, BEIT HAROF'IM, 18 MENUHA VENAHALA STREET, ROOM 27, REHOVOT, 76209, IL

(21) Appl. No.: 11/180,545

(22) Filed: Jul. 14, 2005

(51) Int. Cl. A61B 18/18; A61C 1/00

(52) U.S. Cl. 606/18; 606/10; 433/29; 606/14

(57) ABSTRACT

A laser ablation apparatus including a laser source for generating laser beams in a wavelength range suitable for ablating hard dental tissue, and a scanning device including a beam deflecting element that deflects and scans the laser beams over a surface such that the laser beams impinge on the surface with controllable overlapping (e.g., without overlapping each other). The scanning device and the laser source may be disposed in a hand piece. The scanning device may be coupled to the laser source without an optical fiber or hollow waveguide.

### FIELD OF THE INVENTION

The present invention relates generally to laser ablation, and particularly to a method and apparatus for high speed removal of hard tissue with scanned laser energy.

### BACKGROUND OF THE INVENTION

Pulsed erbium lasers (e.g., Er:YAG lasers with an emission wavelength of 2.94 μm) have long been used for hard tissue removal. Prior art devices deliver radiation from the laser to the tissue by fiber (or hollow waveguide). A problem with the prior art is that the quality of the laser beam decreases after passing through the fiber as compared to the original beam, and the beam cannot be focused to a spot much smaller than the fiber diameter. Conical tips have been used to decrease the spot size on the tissue, but such tips also have inherent energy losses and the beam exits the tip with a large divergence angle. Consequently, it is impossible to drill a cylindrical hole with such a conical tip and additional healthy tooth material is unnecessarily ablated.

Because of the relatively large spot size in current prior art systems, the laser shots overlap each other almost 100% of the time (referred to as a fill factor of almost 100%). Overlapping pulses give rise to another problem. Each pulse leaves a layer of dry hydroxide apatite, which has a low absorption for laser energy, thereby slowing the ablation speed (“first pulse effect”). With a pulse repetition rate of about 5-50 Hz in current prior art systems, there is no time to rehydrate the layer, even with a water spray. Some laser manufacturers recommend moving the tip in the XY plane during treatment in order to decrease the overlapping, but this leads to an increased removal of healthy tissue and also decreases overall speed. It can also lead to potentially harmful temperature increases in the pulp chamber.

The prior art has tried to solve this problem with different techniques for water cooling. For example, the article “Scanning ablation of dental hard tissue with erbium laser radiation”, M. Zeck et al., Proc. SPIE Vol. 2623, p. 94-102, Medical Applications of Lasers III, Frederic Laffitte; Raimund Hibst; Hans-Dieter Reidenbach; Herbert J. Geschwind; Pasquale Spinelli; Marie-Ange D'Hallewin; J. A. Carruth; Giulio Maira; Guilhem Godlewski; Editors, January 1996, discusses using an Er:YAG laser to ablate hard dental tissues. They found that the creation of unwanted “recrystallizations” depended on a complex dependence of spot size, energy density, quantity of spray cooling and pulse duration.

### SUMMARY OF THE INVENTION

As described more in detail hereinbelow, the present invention seeks to provide a method and apparatus for hard tissue removal or ablation, which may increase the speed of hard tissue removal, minimize the removal of healthy tissue, and decrease the pain level.

There is provided in accordance with an embodiment of the present invention laser ablation apparatus including a laser source for generating laser beams in a wavelength range suitable for ablating hard dental tissue, and a scanning device including a beam deflecting element that deflects and scans the laser beams over a surface such that the laser beams impinge on the surface with controllable overlapping (e.g., without overlapping each other). The scanning device and the laser source may be disposed in a hand piece. The scanning device may be coupled to the laser source without an optical fiber or hollow waveguide.

The scanning device may include one or more movable optical elements that move in a linear or rotational direction, such as but not limited to, a wedge, a tilted flat parallel plate, a mirror, and an off-centered lens and any combination thereof.

### BRIEF DESCRIPTION OF THE DRAWINGS

The present invention will be understood and appreciated more fully from the following detailed description, taken in conjunction with the drawings in which:

FIGS. 1A and 1B are simplified illustrations of laser scans performed in accordance with embodiments of the present invention; and

FIG. 2 is a simplified schematic illustration of laser ablation apparatus, constructed and operative in accordance with an embodiment of the present invention.

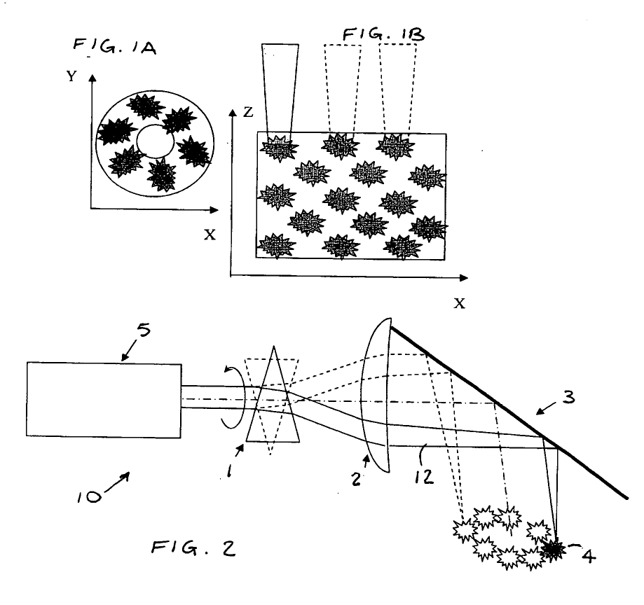

### DETAILED DESCRIPTION OF EMBODIMENTS

Reference is now made to FIG. 2, which illustrates laser ablation apparatus 10, constructed and operative in accordance with an embodiment of the present invention.

The laser ablation apparatus 10 may include a laser source 5 for generating laser beams 12 in a wavelength range suitable for ablating hard dental tissue. For example, the laser beams 12 may have a wavelength in a range of 2700 nm-3000 nm. In a preferred embodiment, the laser source 5 may be an erbium laser, e.g., an Er:YAG laser with an emission wavelength of 2940 nm. The laser source 5 may have a beam quality of at least M²=10 or 30, and may have an energy level in a range of 0.01-2 J at the target surface. (As is known in the art, the beam quality M² is the ratio of the laser beam's multimode diameter-divergence product to the ideal diffraction limited (TEM₀₀) beam diameter-divergence product.) In one embodiment, the laser source 5 may be pulsed and have a frequency level in a range of 1-200 Hz. In another embodiment, the laser source 5 may be continuous wave and have a power level in a range of 0.1-50 W at the target surface.

A scanning device 1, which may include a beam deflecting element, deflects and scans the laser beams 12 over a surface such that the laser beams 12 impinge on the surface with controllable overlapping. For example, beam deflecting element is operative to deflect and scan the laser beams over the surface such that the laser beams impinge on the surface without overlapping each other (e.g., without overlapping each other)without overlapping each other. In accordance with an embodiment of the present invention, the scanning device 1 may include one or more rotating optical elements, such as but not limited to, a wedge, a tilted flat parallel plate, a mirror, and/or an off-centered lens and any combination thereof. As the optical element rotates, it deviates or deflects the laser beam 12 about the mechanical rotating axis. The laser beam 12 may then pass through one or more lenses 2 and be reflected off a mirror 3 (e.g., a folding mirror) to produce a predefined spot 4 on the treatment area. The laser beam 12 may have a diameter at the target site in a range of 0.05 mm-1 mm, for example.

A more complicated design may include two or more rotating optical elements as a scanning device. In this manner, more complicated scanning patterns may be produced.

The scanning device 1 and the laser source 5 may be disposed in a hand piece. The scanning device 1 may be coupled to the laser source 5 without an optical fiber or hollow waveguide.

In one non-limiting method of using laser ablation apparatus 10, a high quality laser beam 12 is focused down to a small spot size 4. The spot is scanned over a target surface, e.g., for removal area of tooth (or bone), with a fill factor less than 100% (e.g., around 50%). Ablation of the hard tissue is associated with explosions at and slightly below the tissue surface. Due to these explosions, areas of tissue outside the radiated zones are also ablated. After scanning a first area, the next scan (next layer) is spatially shifted (by scanning device 1) so that the laser beam 12 will not impinge on the same place as the first area. In this manner, the time between laser beam shots impinging on the same place is increased which leaves more time for hydration of a dry layer and therefore improves the ablation efficiency.

Each pulse or laser beam shot can be of low energy to decrease the pain level (one of the origins of pain is mechanical stress due to shock waves). As in the prior art, water or other cooling fluids may be used to irrigate the ablation site for cooling. With the present invention, the small spot size increases the drilling or ablating efficiency and does not heat the tissue as much as the prior art, leading to lower temperatures and better cooling efficiency.

The speed of rotation of the optical element may be selected as a function of the pulse repetition rate in order to allow the distance between shots to be equal to or less than the spot diameter. Alternatively, the pulse repetition rate may be a function of the rotation speed. The rotation speed and the pulse repetition rate may not be synchronized and the repetition rate may be varied a little bit in order to avoid shooting on the same spot, and to control the fill factor. In such a manner, the laser beams may be deflected and impinge on the surface with controllable overlapping.

It will be appreciated by persons skilled in the art that the present invention is not limited by what has been particularly shown and described hereinabove. Rather the scope of the present invention includes both combinations and subcombinations of the features described hereinabove as well as modifications and variations thereof which would occur to a person of skill in the art upon reading the foregoing description and which are not in the prior art.

What is claimed is:

1. Laser ablation apparatus comprising:
a laser source for generating laser beams in a wavelength range suitable for ablating hard dental tissue; and
a scanning device comprising a beam deflecting element that deflects and scans the laser beams over a surface such that the laser beams impinge on the surface with controllable overlapping.

2. Laser ablation apparatus according to claim 1, wherein said beam deflecting element is operative to deflect and scan the laser beams over the surface such that the laser beams impinge on the surface without overlapping each other.

3. Laser ablation apparatus according to claim 1, wherein said scanning device and said laser source are disposed in a hand piece.

4. Laser ablation apparatus according to claim 3, wherein said scanning device is coupled to said laser source without an optical fiber.

5. Laser ablation apparatus according to claim 3, wherein said scanning device is coupled to said laser source without a hollow waveguide.

6. Laser ablation apparatus according to claim 1, wherein said laser beams have a wavelength in a range of 2700 nm-3000 nm.

7. Laser ablation apparatus according to claim 1, wherein said laser source comprises an erbium laser.

8. Laser ablation apparatus according to claim 1, wherein said laser source has a beam quality of at least M²=10.

9. Laser ablation apparatus according to claim 1, wherein said laser source has a beam quality of at least M²=30.

10. Laser ablation apparatus according to claim 1, wherein said laser source has an energy level in a range of 0.01-2 J at said surface.

11. Laser ablation apparatus according to claim 1, wherein said laser source is pulsed and has a frequency level in a range of 1-200 Hz.

12. Laser ablation apparatus according to claim 1, wherein said laser source is continuous wave and has a power level in a range of 0.1-50 W at said surface.

13. Laser ablation apparatus according to claim 1, wherein said scanning device comprises at least one rotating optical element.

14. Laser ablation apparatus according to claim 13, wherein said at least one rotating optical element comprises at least one of a wedge, a tilted flat parallel plate, a mirror, and an off-centered lens.

15. Laser ablation apparatus according to claim 1, further comprising at least one lens for focusing the laser beams.

16. Laser ablation apparatus according to claim 1, further comprising a folding mirror that reflects the laser beams exiting said scanning device towards said surface.
